SEQUENCE LISTING (5 sequences)

SEQ ID NO: 1 - RNA, 28 nt, Artificial Sequence
  5′-end adapter
  misc_feature (21)..(28): n is a, c, g, or u
acacgacgcucuuccgaucunnnnnnnn

SEQ ID NO: 2 - DNA, 21 bp, Artificial Sequence
  3′-end adapter
agatcggaagagcacacgtct

SEQ ID NO: 3 - DNA, 21 bp, Artificial Sequence
  a primer for the reverse transcription
agacgtgtgctcttccgatct

SEQ ID NO: 4 - DNA, 58 bp, Artificial Sequence
  P5 primer
aatgatacggcgaccaccgagatctacactctttccctacacgacgctcttccgatct

SEQ ID NO: 5 - DNA, 58 bp, Artificial Sequence
  P3 primer
caagcagaagacggcatacgagatgtgactggagttcagacgtgtgctcttccgatct

(19) United States
(12) Patent Application Publication
(10) Pub. No.: US 2024/0076735 A1
(43) Pub. Date: Mar. 7, 2024

(54) METHOD FOR DETECTING WHOLE TRANSCRIPTOME RNA STRUCTURE AND USE THEREOF

(71) Applicant: TSINGHUA UNIVERSITY, Beijing (CN)

(72) Inventors: Qiangfeng ZHANG, Beijing (CN); Jinsong ZHANG, Beijing (CN)

(21) Appl. No.: 18/260,442

(22) PCT Filed: Nov. 16, 2020
(86) PCT No.: PCT/CN2020/128949

§ 371 Date: Jul. 5, 2023

(51) Int. Cl. C12Q 1/6874; C12Q 1/6858

(52) U.S. Cl. CPC C12Q 1/6874; C12Q 1/6858; C12Q 2600/156; C12Q 2600/16; C12Q 2600/178

(57) ABSTRACT

A method for detecting a whole transcriptome RNA structure and the use thereof A method for detecting an intact RNA structure by combining in vivo click chemistry selective 2′-hydroxy acylation and mutation map analysis. Combined with the RNA immunoprecipitation technology, the method is further applied to analyze a RNA structure map of Dicer-bound substrates, and reveals the structural type and characteristics of the Dicer substrates. The method provided for detecting a whole transcriptome RNA structure can also perform complete full-length structure analysis on small RNAs, thereby laying the foundation for the research on the structure and biological functions of RNA molecules in a whole transcriptome in cells.

### TECHNICAL FIELD

The present invention relates to the technical field of molecular biology, and particularly provides a method for probing whole transcriptome RNA structures and use thereof The present invention can probe the secondary structures of all RNA molecules in cells, especially RNAs of length <200 nt.

### BACKGROUND

Whole transcriptome RNA structure omics combines chemical probing with next-generation sequencing to study RNA structures. Chemical reagents widely used for RNA structure probing in vivo include dimethyl sulfate (DMS), 1-methyl-7-nitroisatoic anhydride (1M7), 2-methylnicotinic acid imidazolide-azide (NAI-N3), kethoxal, etc. DMS modifies the N1 and N3 positions of single-stranded adenine and cytosine bases in vivo, whereas NAI-N3 can acrylate the free 2′-hydroxyl groups of all four single-stranded bases. The icSHAPE technology utilizes exactly the NAI-N3's structurally selective 2′-hydroxyl acylation and is combined with subsequent sequencing technology to probe the transcriptome RNA structures. icSHAPE has been used to reveal structural variations of RNAs associated with different biological processes, such as translation, RNA-protein interaction regions and N6-methyladenosine modification sites in living cells. The DMS-seq and icSHAPE technologies are based on the principle that a chemically modified nucleotide generates during reverse transcription a reverse transcription stop signal, by which the probability that the nucleotide is in a single-stranded conformation is determined. However, a limitation of these technologies is that structural information at the 3′ end of a probing target will be missing due to the difficulty of aligning short sequencing reads produced at the 3′ end. It may be an intact transcript or a fragment thereof in the study, for example, a functional region of a long RNA, that will be missing. Such a technical deficiency severely restricts the structural analysis of small-fragment targets, such as small RNAs (sRNAs, RNAs of length <about 200 nt) or binding sites of RNA-binding proteins (RBPs). The DMS-mutational profiling (DMS-MaPseq) and SHAPE-MaP technologies measure, rather than stop signals, the mutation rates generated during reverse transcription at nucleotides modified by a chemical reagent to overcome the 3′-end structural information missing problem. However, DMS-MaPseq provides partial nucleotide coverage (only adenosine “A” and cytidine “C” nucleotides could be probed), and current SHAPE-MaP reagents (e.g., NMIA and 1M7) have only moderate cell membrane penetration abilities, which limit their RNA structure probing in vivo.

### SUMMARY

In regard to the problems described above, we developed a method for probing whole transcriptome RNA structures. Briefly, we utilize the NAI-N3′s structurally selective modification of RNA 2′-hydroxyl groups in cells and the advantage of mutational profiling in reverse transcription to develop a new structure probing method, icSHAPE-MaP. To demonstrate its capabilities, we use icSHAPE-MaP to determine the complete structural information of cellular sRNAs. In addition, we combine icSHAPE-MaP with RNA immunoprecipitation (RIP) to determine the structural profile of substrates of the RNA endonuclease Dicer in terms of the overall picture.

Using the method for probing RNA structures proposed by the present invention—icSHAPE-MaP—and tertiary structural modeling, we found that spatial distance is an important parameter in Dicer's pre-miRNA processing.

To solve the above problems in the prior art, the present invention provides a method for probing whole transcriptome RNA structures. According to the present invention, the structural profile of substrate RNAs bound by Dicer is successfully parsed, and the structural types and characteristics of Dicer substrates are revealed. The present invention provides a method for probing nucleic acid structures and use thereof, the method comprising: 1) modifying a nucleic acid with a labeling reagent; 2) processing the nucleic acid; 3) sequencing the processed nucleic acid; 4) calculating structure scores according to the sequencing result; and 5) predicting a structure of the nucleic acid. The nucleic acid is an RNA; further, the RNA is a full-length RNA; still further, the RNA is a transcriptome RNA; still further, the RNA is a small RNA; still further, the RNA may be a miRNA, a snoRNA, a snRNA, a tRNA, a vault RNA, a Y RNA, a pre-miRNA, a miscRNA, a 5S rRNA, etc., or an RNA transcript fragment, such as an exon and an intron of an mRNA, an exon and an intron of a lncRNA, etc. In one specific embodiment, the present invention provides a method for probing RNA structures, which comprises: 1) modifying a nucleic acid with a labeling reagent; 2) processing the RNA; 3) sequencing the product of the processing; 4) calculating structure scores according to the sequencing result; and 5) predicting a structure of the nucleic acid.

Further, the method for probing RNA structures comprises one of steps a)-d):
a) in step 2), the processing is reverse transcription of the RNA to obtain a cDNA;
b) in step 3), the product of the processing is a cDNA, and the sequencing is deep sequencing of the cDNA;
c) in step 4), calculating structure scores comprises steps of determining the frequency of mutations for each nucleotide site and calculating mutation rates; and
d) in step 5), predicting the structure of the nucleic acid comprises using the RNA structure score profile obtained in step 4) in predicting a secondary structure, a tertiary structure or other higher-order structures of the RNA.

In one specific embodiment, the present invention provides a method for probing whole transcriptome RNA structures, which comprises: 1) modifying a nucleic acid with a labeling reagent; 2) processing the RNA; 3) sequencing the product of the processing; 4) calculating structure scores according to the sequencing result; and 5) predicting a structure of the nucleic acid.

Further, the method for probing whole transcriptome RNA structures comprises one of steps a)-d):
a) in step 2), the processing is reverse transcription of the RNA to obtain a cDNA;
b) in step 3), the product of the processing is a cDNA, and the sequencing is deep sequencing of the cDNA;
c) in step 4), calculating structure scores comprises steps of determining the frequency of mutations for each nucleotide site and calculating mutation rates; and
d) in step 5), predicting the structure of the nucleic acid comprises using the RNA structure score profile obtained in step 4) in predicting a secondary structure, a tertiary structure or other higher-order structures of the RNA.

Preferably, the secondary structure includes single-stranded RNAs, paired double-stranded RNAs, stem loops or hairpins, bulge loops and contacts or multi-branched loops, internal loops, pseudoknots, kissing hairpins, etc. The tertiary structure is a complex structure resulting from the further folding of the nucleic acid chain of the RNA molecule in spatial conformation based on the secondary structure. The other higher-order structures include spatial conformations of RNA-protein complexes and the like.

In the method for probing whole transcriptome RNA structures provided by the present invention, the structure probing method may be DMS-mutational profiling or SHAPE-MaP (mutational profiling).

Further, the labeling reagent is a chemical modification reagent. Preferably, the chemical modification reagent has high intracellular reactivity. The high intracellular reactivity refers to the ability to selectively react with nucleotides of structures that are more single-stranded in RNAs in a cell to generate sufficient modification sites within a reasonable time. Examples include NAI, NAIN3, DMS and kethoxal. In contrast, 1M7 and NMIA are modification reagents with low intracellular reactivity.

Preferably, the labeling reagent is dimethyl sulfate (DMS), 2-methylnicotinic acid imidazolide-azide (NAI-N3) or kethoxal; more preferably, the labeling reagent is 2-methylnicotinic acid imidazolide-azide (NAI-N3).

Further, the method can probe all types of RNA structures in cells in vivo or in vitro. Still further, the RNA may be 200 nt or less in length.

In the method for probing whole transcriptome RNA structures provided by the present invention, modifying the nucleic acid with the labeling reagent in step 1) is specifically: co-incubating cells with the labeling reagent, and then extracting RNA; or mixing in vitro RNA with the labeling reagent, and then purifying and extracting RNA with a kit.

Further, 5′- and 3′-end adapters are ligated to the chemically modified RNA prior to reverse transcription.

Still further, the 5′-end adapter has the following genetic sequence: 5′-rArCrArCrGrArCrGrCrUrCrUrUrCrCrGrArUrCrUrNrNrNrNrNrNrNrN-3′ (SEQ ID No. 1), and the 3′-end adapter has the following genetic sequence: 5′ adenylated-AGATCGGAAGAGCACACGTCT-3′ (SEQ ID No. 2) SpacerC3.

Further, a primer for the reverse transcription has the genetic sequence of

(SEQ ID NO. 3)
5′-AGACGTGTGCTCTTCCGATCT-3′.

In the method for probing whole transcriptome RNA structures provided by the present invention, the cDNA obtained in step 2) is added to a PCR system for amplification reactions, and the resulting PCR product is subjected to deep sequencing.

Further, the PCR system comprises: a P5 primer, a P3 primer, 25× SYBR Green, and 2× Phusion High-Fidelity PCR master mix.

Still further, the P5 primer has the genetic sequence of 5′-AATGATACGGCGACCACCGAGATCTACACTCTTTCCCTACACGACGCTC TTCCGATCT-3′ (SEQ ID No. 4), and the P3 primer has the genetic sequence of 5′-CAAGCAGAAGACGGCATACGAGAT [8-base barcode]GTGACTGGAGTTCAGACGTGTGCTCTTCCGATCT-3′ (SEQ ID No. 5). An 8-base barcode is inserted between “ . . . GAGAT” and “GTGAC . . . ”.

Still further, in the sequence of the P3 primer, the 8-base barcode is used to distinguish between sequencing libraries generated from different samples.

Still further, the PCR program is as follows: stage I: 98° C. for 1 min; stage II: 98° C. for 15 s, 65° C. for 30 s, and 7 ° C. for 45 s; stage II is repeated several times. The number of repetitions is determined according to the fluorescence value displayed on a qPCR instrument, and is generally 13-15.

In the method for probing whole transcriptome RNA structures provided by the present invention, a threshold value of sequencing coverage may be 1000× or 500×, and is preferably 2000×.

Further, in step 4), calculating the assignment includes any one of the following steps:
a) pre-processing sequencing data, comprising: removing 3′ adapters (preferably with Cutadapt), filtering high-quality reads (preferably with Trimmomatic), and deleting duplicate sequences (preferably with Peri);
b) mapping clean reads to a reference sequence (preferably with STAR);
c) calculating icSHAPE-MaP structure scores (preferably with Shapemapper2);
d) predicting a secondary structure of the RNA (preferably with RNAstructure package); and
e) visualizing the secondary structure of the RNA (preferably with VARNA).

Further, the RNA sequence is an sRNA sequence or a protein-bound RNA. Further, in calculating icSHAPE-MaP structure scores, the mutation rates involve all mutation types, such as mismatch, insertion, deletion and other complex mutations.

Further, a mutation rate is calculated for each nucleic acid with shape_mutation_counter.

Still further, the icSHAPE-MaP structure score for base i is calculated by the following formula:

$${s}_{i} = \frac{{r\_nai}_{i} - {r\_dmso}_{i}}{f}$$

wherein r represents a mutation rate, nai represents a labeling reagent sample group, dmso represents a DMSO sample group, and f represents a normalization factor.

The present invention further provides a method for probing specific RNA structures, which combines the method described above with RNA immunoprecipitation.

Further, the specific RNA is a protein-bound RNA, such as a Dicer-bound substrate RNA.

The present invention further provides a kit for probing whole transcriptome RNA structures, wherein the kit comprises the chemical modification reagent and the nucleotide sequences described in any one of the methods for probing whole transcriptome RNA structures described above.

The present invention has the following beneficial effects:

The present invention proposes a new biotechnology, “icSHAPE-MaP”, which probes intact RNA secondary structures in vivo by utilizing mutational profiling of reverse transcriptase to detect modifications induced by a labeling reagent with high intracellular reactivity, such as NAI-N3. Importantly, this method allows for structural analysis of small-sized RNA species (full-length sRNAs or fragments (e.g., RBP-binding sites) of long RNAs). The present invention also demonstrates use of icSHAPE-MaP in revealing the structural profile of Dicer substrate sRNAs. In the future, icSHAPE-MaP can be used to reveal the structural features of RNAs bound by other RBPs.

The foregoing is merely a summary of some aspects of the present invention, and is not, and should not be construed as, limiting the present invention in any way. Unless otherwise specified, the practice of the present invention will adopt traditional techniques of cell biology, cell culture, molecular biology, immunology, and the like. These techniques are explained in detail in the following documents. For example:
1. Reuter, J. S., and Mathews, D. H. (2010). RNAstructure: software for RNA secondary structure prediction and analysis. BMC Bioinformatics 11, 129. 16.

2. Das, R., Karanicolas, J., and Baker, D. (2010). Atomic accuracy in predicting and designing noncanonical RNA structure. Nat Methods 7, 291-294 23.

3. Zubradt, M., Gupta, P., Persad, S. et al. DMS-MaPseq for genome-wide or targeted RNA structure probing in vivo. Nat Methods 14, 75-82 (2017). https://doi.org/10.1038/nmeth.4057.

4. Siegfried, N., Busan, S., Rice, G. et al. RNA motif discovery by SHAPE and mutational profiling (SHAPE-MaP). Nat Methods 11, 959-965 (2014). https://doi.org/10.1038/nmeth.3029.

### BRIEF DESCRIPTION OF THE DRAWINGS

To more clearly illustrate the examples of the present invention or the existing technical solutions, the drawings needed in the description of the examples or the prior art are briefly described below. It is obvious that the drawings in the description below are only some examples described in the present invention and those skilled in the art can acquire other drawings according to these drawings without creative efforts.

FIG. 1 shows the proportions of reads aligned to a reference genome that carry mutations in different groups. The control group, i.e., the DMSO group, has a significantly lower proportion of reads carrying mutations than the in vivo NAI-N3 modification group and the in vitro NAI-N3 modification group, which indicates that the increase in the mutation rate is really caused by the NAI-N3 modification.

FIG. 2 is a bar chart of mutation rates of four bases in samples of the control DMSO group, the in vivo NAI-N3 modification group and the in vitro NAI-N3 modification group, which shows that NAI-N3 can simultaneously modify the four bases.

FIG. 3 shows the mutation rates of eight types of mutations in human 5S rRNA, which indicate the contribution of the different types of mutations to the mutation rates.

FIG. 4 shows the mutation rate of each nucleotide position in the control DMSO group and the in vivo NAI-N3 modification group of human 5S rRNA.

FIG. 5 shows the known secondary structure model of 5S rRNA and the icSHAPE-MaP structure score of each nucleotide.

FIG. 6 shows examples of one snoRNA and one tRNA, as well as the secondary structure models thereof, with icSHAPE-MaP structure scores.

FIG. 7 shows the cumulative distribution curve for the Pearson correlation coefficient of mutation rates of the NAI-N3 modification group for the same region between two replicates.

FIG. 8 shows the cumulative distribution curves for the Pearson correlation coefficient of mutation rates in samples of the DMS modification group (upper) or the NAI-N3 modification group (lower), for the A and C bases, between two replicates.

FIG. 9 is a ring diagram of different types of Dicer substrates.

FIG. 10 is a violin plot showing the length distribution of Dicer-bound fragments.

FIG. 11 shows the read coverage of two fragments in GDI1 and DICER1 enriched by RNA immunoprecipitation.

FIG. 12 is a ring diagram of different types of RNA with icSHAPE-MaP structure scores.

FIG. 13 is a heatmap of the Pearson correlation coefficient of mutation rates of nucleotides between two replicates in DMSO or NAI-N3 samples.

FIG. 14 is a ring diagram of different types of Dicer-enriched RNA with icSHAPE-MaP structure scores.

FIG. 15 is a bar chart of the relative expression level of the top 150 pre-miRNAs in HEK293T cells, where height indicates the relative expression level of pre-miRNAs.

FIG. 16 is a violin plot of area under curves (AUCs) obtained by comparison of 209 tRNA secondary structures from the GtRNAdb database with the resulting icSHAPE-MaP structure scores.

FIG. 17 shows the secondary structure model of hsa-miR-125a constructed by RNAstructure software with structure scores as constraints; the color of each nucleotide represents an icSHAPE-MaP structure score level.

FIG. 18 shows the secondary structure model of hsa-miR-125a from the miRBase database; similarly, the color of each nucleotide represents an icSHAPE-MaP structure score level.

FIG. 19 shows the secondary structure models of hsa-miR-19a and hsa-miR-27b pre-miRNA, as well as their corresponding icSHAPE-MaP structure score for each nucleotide. The upper models are constructed by RNAStructure software with their icSHAPE-MaP structure scores as constraints; the lower models are from the miRBase database.

FIG. 20 is a violin plot of pseudo-free energy between 108 pre-miRNA structures from the miRBase database or predicted by RNAStructure.

### DETAILED DESCRIPTION

The present invention is described below in further detail through examples. However, this should not be understood as limiting the scope of the above-described subject matter of the present invention to the following examples. Various substitutions and alterations made according to the general knowledge and practice in the art without departing from the technical concepts of the present invention described above should fall within the scope of the present invention.

The experimental methods used in the following examples are all conventional methods unless otherwise specified. The materials, reagents, etc., used in the following examples are all commercially available unless otherwise specified.

### EXAMPLE 1

### Cell Culture and Transfection

The HEK293T cell line was purchased from ATCC. The Dicer KO HEK293T cell line (NoDice 2-20) was a gift from Dr. Bryan R. Cullen of Duke University. Cells were cultured in high-glucose DMEM containing L-glutamine, sodium pyruvate (Thermo Scientific HyClone) and 10% fetal bovine serum in a 37° C., 5% CO₂ humidified incubator. All cell transfection experiments were carried out using polyethyleneimine (PEI) (Sigma-Aldrich). Example 2. Chemical Modification of RNA HEK293T cells were scraped off from culture dishes and washed with PBS. The cells were resuspended in 100 mM NAI-N3 and incubated at 37° C. on a Thermomixer for 5 min. After the mixture was centrifuged at 2500 g at 4° C. for 1 min, the reaction was stopped, and the supernatant was subsequently removed. The cells were collected and resuspended in 250 μL of PBS, and 750 μL of TRIzol LS reagent was added for RNA extraction according to the instructions. The resulting RNAs or RNAs prepared in vitro were screened by size (25-200 nt) on a 6% denaturing urea-PAGE gel. The gel containing RNAs of specific length was crushed, then placed in a buffer (500 mM NaCl, 1 mM EDTA pH 8.0, 10 mM Tris-HCl pH 8.0), and incubated with rotation at 4° C. overnight. The solution containing eluted RNAs was centrifuged using a 0.45 μm Spin-X column (Thermo Fisher), concentrated, and purified with an RNA concentration kit (Zymo) to obtain RNAs of specific size (25-200 nt).

### EXAMPLE 3

### RNA Immunoprecipitation

NoDice 2-20 cells were transfected with a plasmid expressing human Dicer deprived of cleavage activity (containing two mutations (D1320A and D1709A) in its RNase III domains, Addgene). 9×10 6 cells were seeded into a 15-cm plate on the first day. After 24 h, the cells were transfected with 20 μg of plasmids and 60 μL (1 μg/μL) of PEI. Specifically, the plasmids and PEI were separately mixed with 1 mL of Opti-MEM I reduced serum medium (Gibco) first and incubated. Then the two mixtures were mixed, left at room temperature for 15 min, and then added to cells. After 48 h, the cells were lysed with a lysis buffer. The lysis buffer was prepared using 50 mM Tris-HCl pH 7.4, 150 mM NaCl, 1% Triton-X 100 and 1 mM EDTA and was supplemented with the proteinase inhibitor cocktail (Roche) and RNase inhibitor RiboLock (40 U/mL, Thermo Fisher). The lysate was centrifuged at 15,000 g for 10 min at 4° C. to remove insoluble cell debris. The supernatant was incubated with anti-FLAG M2 magnetic beads (Sigma) at room temperature for 3 h.

After incubation, the magnetic beads were washed once with a high salt wash buffer (50 mM Tris-HCl pH 7.4, 1 M NaCl, 1% Triton-X 100, proteinase inhibitor cocktail (Roche), RiboLock (Thermo Fisher, 40 U/mL)) and twice with a low salt wash buffer (50 mM Tris-HCl pH 7.4, 150 mM NaCl, 5 mM EDTA, proteinase inhibitor cocktail (Roche), RiboLock (Thermo Fisher, 40 U/mL)). After the last wash, the beads were incubated with a modification buffer (333 mM HEPES, 20 mM MgCl2, 150 mM NaCl, 50 mM NAI-N3) on a Thermomixer at 37° C. at 1000 rpm for 12 min (NAI-N3 modification group). For the control DMSO group, NAI-N3 was replaced by DMSO in the modification buffer. Finally, RNAs were extracted with Trizol LS.

### EXAMPLE 4

### Library Construction

The RNAs (8 jut) obtained in Example 2 or Example 3 were ligated with a 3′ linker by incubation with a 3′ ligation reaction mixture (6 μL of PEG8000, 1 μL of 3′ linker (10 μM), 1 μL of DTT (100 mM), 2 μL of 10× ligation buffer, 1 μL of T4 RNA ligase KQ (NEB), 1 μL of RiboLock) at 25° C. for 2 h. Then the enzyme was inactivated at 65° C. for 20 min.

1.2 μL of a reverse transcription primer (10 μM) was added to the above mixture, and the mixture was annealed at 75° C. for 5 min, 37° C. for 15 min and 25° C. for 15 min to pair the reverse transcription primer with the 3′ linker and neutralize the excess 3′ linker.

A 5′ ligation reaction mixture (3 μL of PEG8000, 3μL of 10 mM ATP, 1 μL of 10× ligation buffer, 0.5 jut of RiboLock, 0.5 μL of 5′ linker (20 μM), 1 μL of T4 RNA ligase I (NEB)) was added to the mixture, and the mixture was incubated at 25° C. for 2 h.

The above reaction mixture was purified with an RNA concentration kit (Zymo) to obtain RNAs with 5′ and 3′ linkers linked. 9 μL of a reverse transcription buffer prone to mutate (50 mM Tris-HCl pH 8.0, 500 μM dNTP, 75 mM KCl, 10 mM DTT, 6 mM MnCl2, 1 μL of RiboLock) was added to 10 μL of purified RNAs, and the reaction mixture was incubated at 42° C. for 2 min.

1 μL of SuperScript II (Thermo Fisher) was added to the above reaction mixture, and the mixture was incubated at 42° C. for 3 h for reverse transcription. The cDNA product obtained from the above reaction was purified with a DNA concentration kit (Zymo).

A PCR system was set up with 20 μL of eluted cDNAs and PCR reaction mixture (0.5 μL of P5 primer (20 μM), 0.5 μL of P3 index primer (20 μM), 0.4 μL of 25× SYBR Green, 20 μL of 2× Phusion High-Fidelity PCR master mix (NEB)).

PCR was performed in a qPCR instrument (Agilent, Mx3000P) to monitor the amplification process and was programmed as follows: stage I: 98° C. for 1 min; stage II: 98° C. for 15 s, 65° C. for 30 s, and 72° C. for 45 s; stage II was repeated several times. The number of PCR cycles was determined according to the fluorescence value on the qPCR instrument, and was generally 13-15.

The resulting PCR products were purified with a DNA concentration kit (Zymo), and further size screening (150-330 nt) was performed on a 6% native PAGE gel to remove excess PCR primers. The PCR products were purified from the gel as described earlier to obtain the final PCR products, i.e., the final library. The library was sequenced on the Illumina HiSeq X TEN platform for paired-end 150 cycles.

### EXAMPLE 5

### icSHAPE-MaP Structure Score Calculation

Pre-processing: Adapters were removed with cutadapt (v1.16), low-quality reads were filtered out with Trimmomatic (v0.33), and duplicate sequencing reads in the sequence were removed with a custom Perl script.

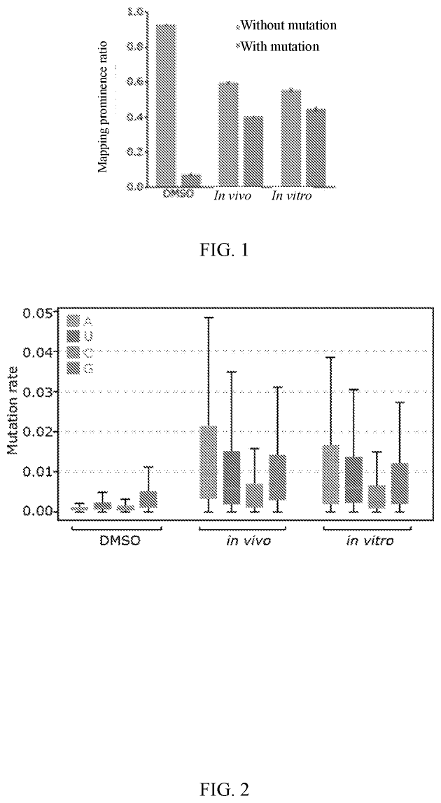

Alignment: Human sRNA sequences less than about 200 nt in length were collected, such as miRNAs (from miRbase v22), snoRNAs (from Gencode v26), snRNAs (from Gencode v26), tRNAs (from GtRNAdb v2.0), vault RNAs (from RefSeq v109), Y RNAs (from RefSeq v109), and 5S rRNAs. The above-processed reads were aligned to the collected human sRNA sequences with STAR (v2.7.1a) using parameters outFilterMismatchNmax=3, outFilterMultimapNmax=10, alignEndsType=Local scoreGap=1000 outSAMmultNmax=1. To find out other sRNA fragments not well annotated on the human genome, the unaligned reads were aligned to the human genome (version GRCh38.p12) to repeat the data analysis described above. The proportion of aligned reads carrying mutations in the NAI-N3 modification group library was significantly increased compared to the control DMSO group library, whether in vivo or in vitro, which indicates that NAI-N3 did cause mutations during reverse transcription (FIG. 1). The increase in the mutation rate was more pronounced at the A and U bases, which agrees with the previous observation that the single-stranded regions were rich in A/U over G/C (FIG. 2).

icSHAPE-MaP structure score calculation: Data between sample replicates were merged (using the merge command of samtools). Shapemapper2 (v2.1.4) was used to calculate final structure scores as follows:
a. Mutations on each read were parsed with shapemapper_mutation_parser. The script counted 8 mutation types: mismatch, insertion, deletion, multi-mismatch, multi-insertion, multi-deletion, complex-insertion, and complex deletion.
b. The frequency of mutations for each nucleotide was counted with shapemapper_mutation_counter.
c. icSHAPE-MaP structure scores were calculated with make_reactivity_profiles.py.
d. The raw structure scores were normalized with normalize_profiles.py.

The calculation process for each base can be briefly summarized by the following formula:

$${s}_{i} = \frac{{r\_nai}_{i} - {r\_dmso}_{i}}{f}$$

The icSHAPE-MaP structure score for base i is the difference between mutation rates of base i in the NAI-N3 modified sample and the control DMSO group sample divided by the normalization factor f.

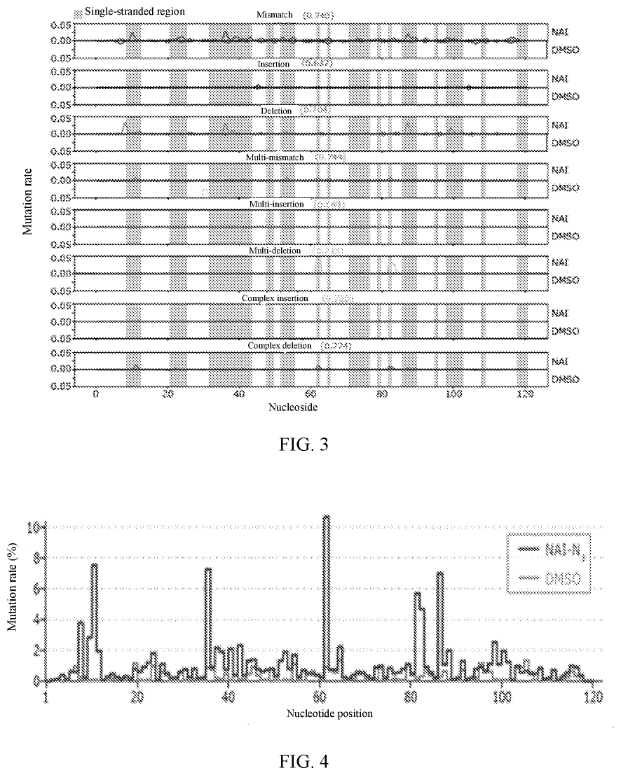

The NAI-N3 modification may cause various types of mutations, including mismatch, insertion, deletion and other complex mutations (FIG. 3).

Correlation of mutation rates between replicates: The total read counts from two replicates were balanced by down-sampling. All bases were sorted by coverage. Bases with coverage greater than 500, 1000, 2000, 3000, 4000 or 5000 were selected to calculate the replicate correlation of mutation rate with sliding window (window size: 50 nt; window step: 10 nt). Finally, a cumulative distribution curve was generated from the correlation data obtained under each threshold.

Computational prediction of RNA secondary structures with constraints: The Fold program in the RNAstructure package (v5.6) was used to predict the secondary structures of RNAs. The icSHAPE-Map structure scores were used as constraints, and the parameters were: -si −0.6 -sm 1.8 -SHAPE icSHAPE-Map.shape-mfe.

Visualization of RNA secondary structures: Secondary structures of RNAs were visualized with the VARNAv3-93 command line. Colors of bases were applied with the parameter “-basesStyle1 on and -applyBasesStyle1 on”.

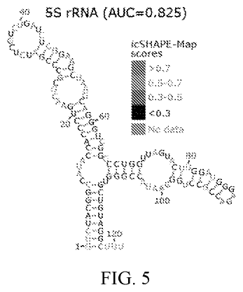

The in vivo structure scores of 186 transcripts and the in vitro structure scores of 250 transcripts were obtained (FIGS. 4-5) by icSHAPE-MaP. The structure scores of 5S rRNAs, AUC=0.825 (the closer to 1, the more consistent) indicate that the obtained structure scores agree well with the known secondary structure model and are very much in line with the known structure, thereby demonstrating the accuracy of icSHAPE-MaP (see FIG. 5).

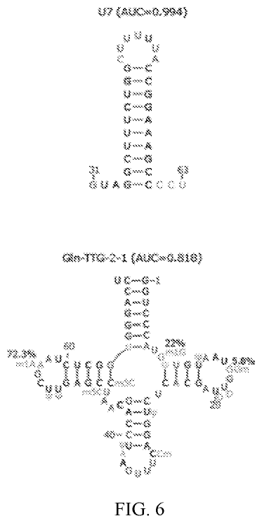

Accurate structure scores for other sRNAs with known secondary or tertiary structure models were obtained by icSHAPE-MaP, including the 3′ fragments of RNU7 (small nuclear RNAs, snRNAs, AUC=0.994) and Gln-TTG-2-1 (tRNAs, AUC=0.818) (FIG. 6).

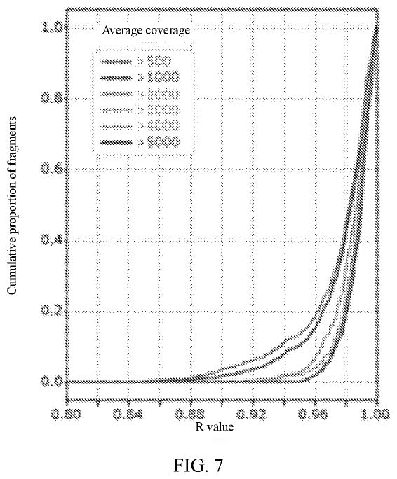

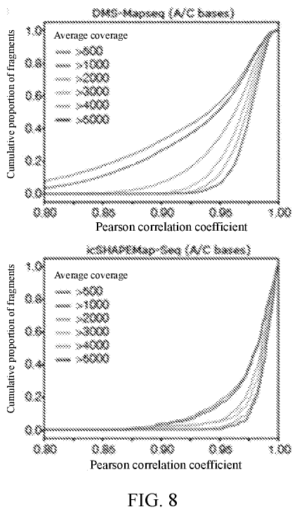

The sequencing coverage threshold of 2000× yielded very high-quality, high-reproducibility structure scores. When considering the tradeoff between sequencing costs, data quality and reproducibility, it can be seen that with 500 as the sequencing coverage threshold, the Pearson correlation coefficient of mutation rates of more than 80% of the fragments was greater than 0.96, which indicates that the reproducibility of our experiments was good and that 1000× or even 500× coverage can be a reasonable threshold (FIG. 7). Importantly, with the same sequencing coverage threshold, the NAI-N3 modification group (icSHAPE-MaP technology) showed better reproducibility than the DMS modification group (DMS-MaPseq technology). From the viewpoint of sequencing costs, the icSHAPE-MaP technology requires a shallower depth of sequencing; that is, to meet the same data reproducibility requirement, we found that icSHAPE-MaP required much smaller sequencing coverage than DMS-MaPseq (FIG. 8).

### EXAMPLE 6

### Use of icSHAPE-MaP in Analysis of Dicer Substrates

Dicer belongs to the RNase III family. It cleaves double-stranded RNA (dsRNA) and precursor-microRNA (pre-miRNA) hairpins into mature small interfering RNAs (siRNAs) or microRNAs (miRNAs), respectively. How Dicer precisely determines the cleavage site on its substrates is very important to the generation processes of RNA interference (RNAi) and miRNAs. Studies have shown that Dicer uses different measuring methods to determine its cleavage sites: it measures a certain number of nucleotides, 1) from the 3′ overhang of dsRNA substrates (the 3′ counting rule), 2) or from the phosphate groups of the 5′ ends of pre-miRNAs and dsRNAs (the 5′ counting rule). 3) In addition, in vivo studies of short hairpin RNAs (shRNAs) and pre-miRNAs show that Dicer uses a single-stranded region (a bulge or terminal loop) to precisely anchor 2-nt downstream of the single-stranded region as a cleavage site (the loop counting rule). However, it is unclear when and to what extent these mechanisms apply to pre-miRNA processing. In addition, Dicer can also bind to a variety of substrates without generating corresponding miRNAs or siRNAs, which indicates that it also plays other roles in RNA metabolism. It is unclear whether and how Dicer distinguishes between cleavable and non-cleavable substrates.

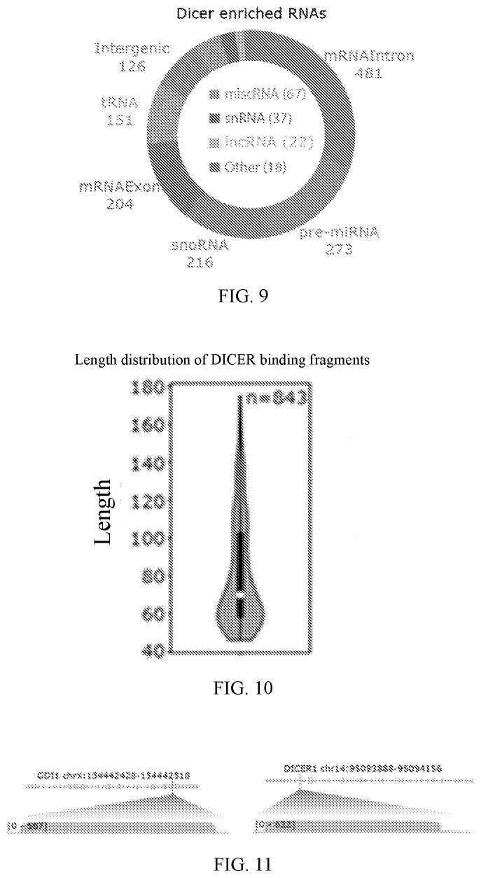

Through the methods described in Examples 1-5, 1595 Dicer-enriched RNAs were detected in the unmodified DMSO group library in the analysis of Dicer substrates (FIG. 9). Compared to other enrichment strategies, the lists of enriched RNAs are highly similar, with more than 50% common pre-miRNAs between them (Table 1).

In addition to pre-miRNAs, we identified other intracellular transcripts having a median length of about 70-nt (FIG. 10), including snoRNAs, tRNAs, intronic and exonic sequence fragments of messenger RNAs (mRNAs), and intergenic region-derived transcripts. The results show that most of the Dicer binding fragments are about 60-70 nt as expected. The read coverage profile of these intronic and exonic fragments shows very clear boundaries, which indicate that they are products formed by mRNA processing at their positions and are, to a certain extent, functional (FIG. 11).

TABLE 1
Comparison of Dicer binding sites found by the
present invention with those found by PAR-CLIP

| | Number of | | | Number of |
|---|---|---|---|---|
| | binding sites | Number of | | binding sites |
| | by RIP | overlap | Rate of | by PAR-CLIP |
| Genotype | method | binding sites | overlap | method |
| pre-miRNA | 273 | 174 | 63.74% | 321 |
| tRNA | 151 | 83 | 54.97% | 369 |
| mRNAExon | 204 | 91 | 44.61% | 3808 |
| lncRNAIntron | 9 | 3 | 33.33% | 85 |
| miscRNA | 65 | 18 | 27.69% | 1140 |
| lncRNAExon | 13 | 3 | 23.08% | 98 |
| intergenic | 126 | 24 | 19.05% | 1238 |
| snRNA | 37 | 6 | 16.22% | 30 |
| mRNAIntron | 481 | 67 | 13.93% | 1237 |
| snoRNA | 216 | 14 | 6.48% | 33 |

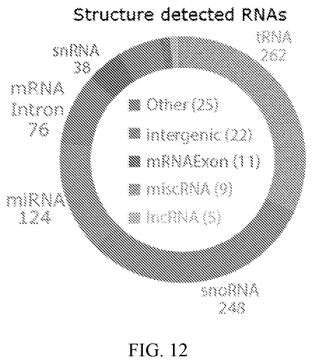

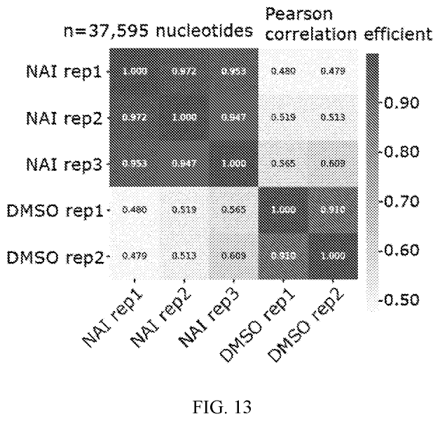

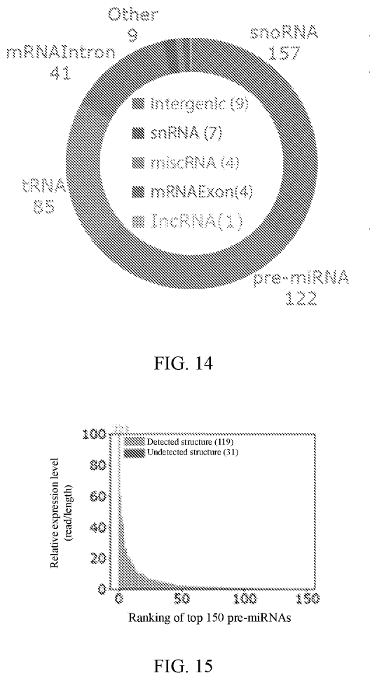

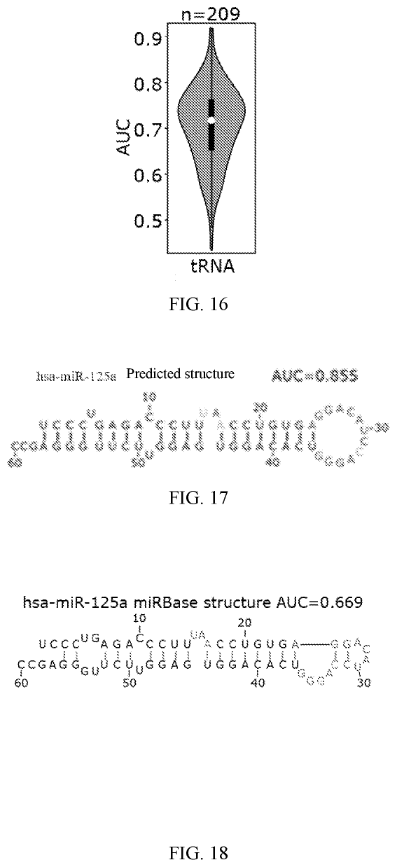

Using RIP-icSHAPE-MaP, we obtained the structural information for 820 well-covered RNAs (>1000× sequencing coverage) (FIG. 12). We found that the mutational profiles were highly correlated within independent biological replicates, which indicates the good reproducibility of the method (FIG. 13). On the basis of our RNA-seq data, we used RIP enrichment scores to classify 439 of the RNAs as Dicer targets, including 122 pre-miRNAs. They included almost all pre-miRNAs with top expression levels in HEK293T cells (FIGS. 14-15). As a structure modeling control, we compared the icSHAPE-MaP structure scores of tRNAs in our dataset to their published structure models in GtRNAdb and calculated AUCs. Most of the AUCs are well above 0.5, with the median above 0.7, which indicates good concordance between the obtained structure scores of most of the tRNAs and the secondary structure models from the GtRNAdb database (FIG. 16) and good agreement between our structure probing and the existing co-evolutionary structure models for tRNAs.

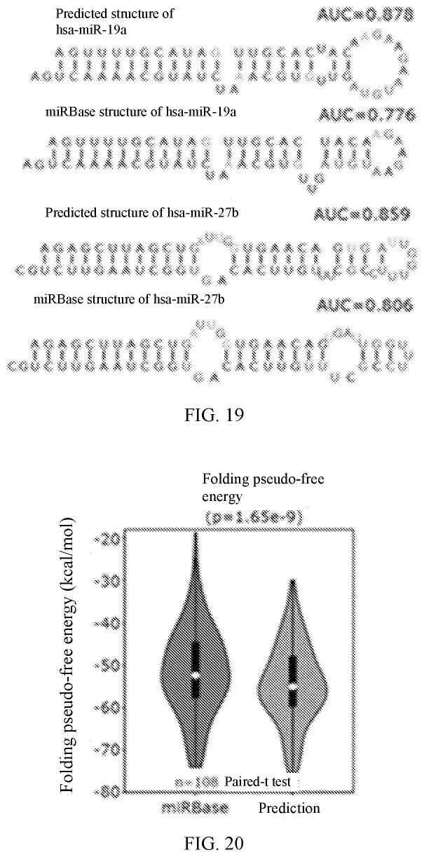

Using the present invention, we obtained a structure model of pre-miR-125a with structure scores as constraints, which contains a 12-nt terminal loop (G25-G36) (FIG. 17). In contrast, the unconstrained structure model from miRbase (version 22.1) indicates that it has multiple bulges, internal loops and a smaller terminal loop (FIG. 18). In addition, the constrained model of pre-miR-19a shows a 12-nt terminal loop, whereas its miRbase model contains a smaller terminal loop, and the constrained model of pre-miR-27b has a 6-nt terminal loop and a nearby bulge, compared to its miRbase model that contains a small 3-nt terminal loop and a nearby large internal loop (FIG. 19). In general, the constrained structure model of pre-miRNAs also has lower free energy than the unconstrained structure model from miRbase (p =1.65e-9). The lower the pseudo-free energy, the more stable the structure. It can be seen that the structure model predicted using RNAstructure with icSHAPE-MaP structure scores as constraints is more stable (FIG. 20). These results indicate that using icSHAPE-MaP structure scores as constraints can precisely simulate RNA secondary structures, which serve as a structural basis for Dicer's processing of its substrates and functional studies.

1. A method for probing nucleic acid structures, wherein the method comprises: 1) modifying a nucleic acid with a labeling reagent; 2) processing the nucleic acid; 3) sequencing the processed nucleic acid; 4) calculating structure scores according to the sequencing result; and 5) predicting a structure of the nucleic acid.

2. The method for probing nucleic acid structures according to claim 1, wherein the nucleic acid is an RNA.

3. The method for probing nucleic acid structures according to claim 2, wherein the nucleic acid is an RNA, and the method comprises one of steps a)-d):
a) in step 2), the processing is reverse transcription of the RNA to obtain a cDNA;
b) in step 3), the product of the processing is a cDNA, and the sequencing is deep sequencing of the cDNA;
c) in step 4), calculating structure scores comprises steps of determining the frequency of mutations for each nucleotide site and calculating mutation rates; and
d) in step 5), predicting the structure of the nucleic acid comprises using the RNA structure score profile obtained in step 4) in predicting a secondary structure, a tertiary structure or other higher-order structures of the RNA.

4. The method for probing nucleic acid structures according to claim 3, wherein the nucleic acid is a whole transcriptome RNA.

5. The method for probing nucleic acid structures according to claim 1, wherein the structure probing method may be DMS-mutational profiling or SHAPE-MaP (mutational profiling).

6. The method for probing nucleic acid structures according to claim 1, wherein the labeling reagent is a chemical modification reagent.

7. The method for probing nucleic acid structures according to claim 2, wherein the method can probe all types of RNA structures in cells in vivo or in vitro.

8. The method for probing nucleic acid structures according to claim 2, wherein the RNA is 200 nt or less in length.

9. The method for probing nucleic acid structures according to claim 1, wherein modifying the nucleic acid with the labeling reagent in step 1) is specifically:
co-incubating cells with the labeling reagent, and then extracting RNA; or mixing in vitro RNA with the labeling reagent, and then purifying and extracting RNA with a kit.

10. The method for probing nucleic acid structures according to claim 3, wherein 5′- and 3′-end adapters are ligated to the chemically modified RNA prior to reverse transcription.

11. The method for probing nucleic acid structures according to claim 10, wherein the 5′-end adapter has the following genetic sequence: 5′-rArCrArCrGrArCrGrCrUrCrUrUrCrCrGrArUrCrUrNrNrNrNrNrNrNrN-3′ (SEQ ID No. 1), and the 3′-end adapter has the following genetic sequence: 5′ adenylated-AGATCGGAAGAGCACACGTCT-3′ (SEQ ID No. 2) SpacerC3.

12. The method for probing nucleic acid structures according to claim 10, wherein a primer for the reverse transcription has the genetic sequence of 5′-AGACGTGTGCTCTTCCGATCT-3′ (SEQ ID No. 3).

13. The method for probing nucleic acid structures according to claim 3, wherein in step 3), the cDNA obtained in step 2) is added to a PCR system for amplification reactions, and the resulting PCR product is subjected to deep sequencing.

14.-16. (canceled)

17. The method for probing nucleic acid structures according to claim 1, wherein in step 4), calculating structure scores comprises any one of the following steps:
a) pre-processing sequencing data, comprising: removing 3′ adapters, filtering high-quality reads, and deleting duplicate sequences;
b) mapping clean reads to a reference sequence;
c) calculating icSHAPE-MaP structure scores;
d) predicting a secondary structure of the RNA; and
e) visualizing the secondary structure of the RNA.

18. (canceled)

19. The method for probing nucleic acid structures according to claim 17, wherein in calculating icSHAPE-MaP structure scores, the mutation rates involve mismatch, insertion, deletion and other complex mutations.

20. The method for probing nucleic acid structures according to claim 17, wherein a mutation rate is calculated for each nucleic acid with shape_mutation_counter.

21. The method for probing nucleic acid structures according to claim 17, wherein the icSHAPE-MaP structure score for base i is calculated by the following formula:
$${s}_{i} = \frac{{r\_nai}_{i} - {r\_dmso}_{i}}{f}$$
wherein r represents a mutation rate, nai represents a labeling reagent sample group, dmso represents a DMSO sample group, and f represents a normalization factor.

22. The method for probing nucleic acid structures according to claim 1, wherein the method further comprises a step of RNA immunoprecipitation to obtain an RNA.

23. The method according to claim 22, wherein the RNA is a protein-bound RNA.

24. A kit for probing whole transcriptome RNA structures, wherein the kit comprises the chemical modification reagent and the nucleotide sequences described in the method for probing nucleic acid structures according to claim 1.
